(19) United States
(12) Patent Application Publication
(10) Pub. No.: US 2008/0173568 A1
(43) Pub. Date: Jul. 24, 2008

(54) Kit suitable for use in a surgical procedure and methods for using the same

(76) Inventor: Mohamed Brageeth, Rotterdam (NL)

Correspondence Address: WITHERS & KEYS, LLC, P. O. BOX 2049, MCDONOUGH, GA, 30253, US

(21) Appl. No.: 11/900,680

(22) Filed: Sep. 13, 2007

Related U.S. Application Data
(60) Provisional application No. 60/846,061, filed on Sep. 20, 2006.

(51) Int. Cl. B65D 85/00; A61B 17/34

(52) U.S. Cl. 206/571; 206/570; 606/185

(57) ABSTRACT

A kit containing various kit components suitable for use in a surgical procedure is disclosed. Methods of using the kit and its components are also disclosed.

### CROSS-REFERENCE TO RELATED APPLICATIONS

This patent application claims the benefit of priority to U.S. provisional patent application Ser. No. 60/846,061 entitled “A KIT SUITABLE FOR USE IN A SURGICAL PROCEDURE AND METHODS FOR USING THE SAME” filed on Sep. 20, 2006, the subject matter of which is incorporated herein in its entirety.

### FIELD OF THE INVENTION

The present invention relates generally to a kit suitable for use in a surgical procedure, and methods of using the kit.

### BACKGROUND OF THE INVENTION

Efforts continue in the art of medical devices to find innovative ways to simplify surgical procedures and reduce costs associated with medical devices used during surgical procedures.

### SUMMARY OF THE INVENTION

The present invention is directed to a kit suitable for use in a surgical procedure, and methods of using the kit. According to one exemplary embodiment of the present invention, the kit comprises a packaging material and components sized to fit within the packaging material, wherein the components comprise a connector that can be used to connect a fluid supply to a trocar, and a balloon. The exemplary kit may contain additional components such as a first attachment member capable of attaching a neck portion of the balloon to a tubular shaft of a trocar, a fluid-supplying device, one or more one-way valves, a trocar seal, and an additional attachment member capable of attaching a cylindrical sleeve portion of a trocar seal to a tubular shaft of a trocar.

The present invention is further directed to methods of using the kit in an operating room setting. In one exemplary embodiment of the present invention, the method of using the kit comprises a method of preparing for a surgical procedure comprising the steps of providing a kit comprising a packaging material and components sized to fit within the packaging material, wherein the components comprise a connector that can be used to connect a fluid supply to a trocar, and a balloon; providing a trocar having a tubular shaft, wherein the tubular shaft has an outer surface suitable for excepting the neck portion of the balloon; removing the components from the packaging material; and assembling the components with the trocar so as to form a device suitable for pumping a fluid into the balloon so as to inflate the balloon. The inflated balloon may be used to separate tissue within a patient during a surgical procedure, such as laparoscopic surgery.

The present invention is even further directed to methods of doing business relating to the kit. In one exemplary embodiment, a method of doing business comprises the steps of providing a kit comprising a packaging material and components sized to fit within the packaging material, wherein the components comprise a connector that can be used to connect a fluid supply to a trocar, and a balloon; wherein the method provides an alternative to commercially available trocar assemblies comprising a pre-assembled trocar/balloon combination. In the disclosed methods of doing business, the kit can be used with any commercially available trocar.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a side view of an exemplary kit of the present invention;

FIG. 2 depicts a side view of an exemplary trocar assembly comprising (i) a trocar and (ii) components of an exemplary kit of the present invention attached to the trocar; and

FIG. 3 depicts a side view of an exemplary trocar assembly comprising (i) a trocar and (ii) components of the exemplary kit shown in FIG. 1 attached to the trocar.

### DETAILED DESCRIPTION OF THE INVENTION

The present invention is directed to kits for use in an operating room setting. The present invention is further directed to methods of using the kit along with any commercially available trocar so as to form a device suitable for pumping a fluid, such as air, into the balloon so as to inflate the balloon. In addition, the present invention is directed to methods of doing business comprising providing a kit use in with a commercially available trocar device.

In one exemplary embodiment of the present invention, the kit comprises a packaging material and components sized to fit within the packaging material, wherein the components comprise a connector that can be used to connect a fluid supply, such as an air supply or water supply (e.g., a conduit for connecting to an air supply or a water supply) to a trocar, and a balloon. In a further exemplary embodiment, the kit comprises the above-described components, wherein the components further comprise a first attachment member capable of attaching a neck portion of the balloon to a tubular shaft of a trocar. In yet a further exemplary embodiment, the kit comprises the above-described components, wherein the components further comprise a syringe (i.e., the fluid-supplying device), and a first one-way valve. In yet a further exemplary embodiment, the kit comprises the above-described components, wherein the connector comprises a three-way connector, and the components further comprise a syringe, and two one-way valves. In all of the exemplary embodiments, desirably, the kit is a sterile kit.

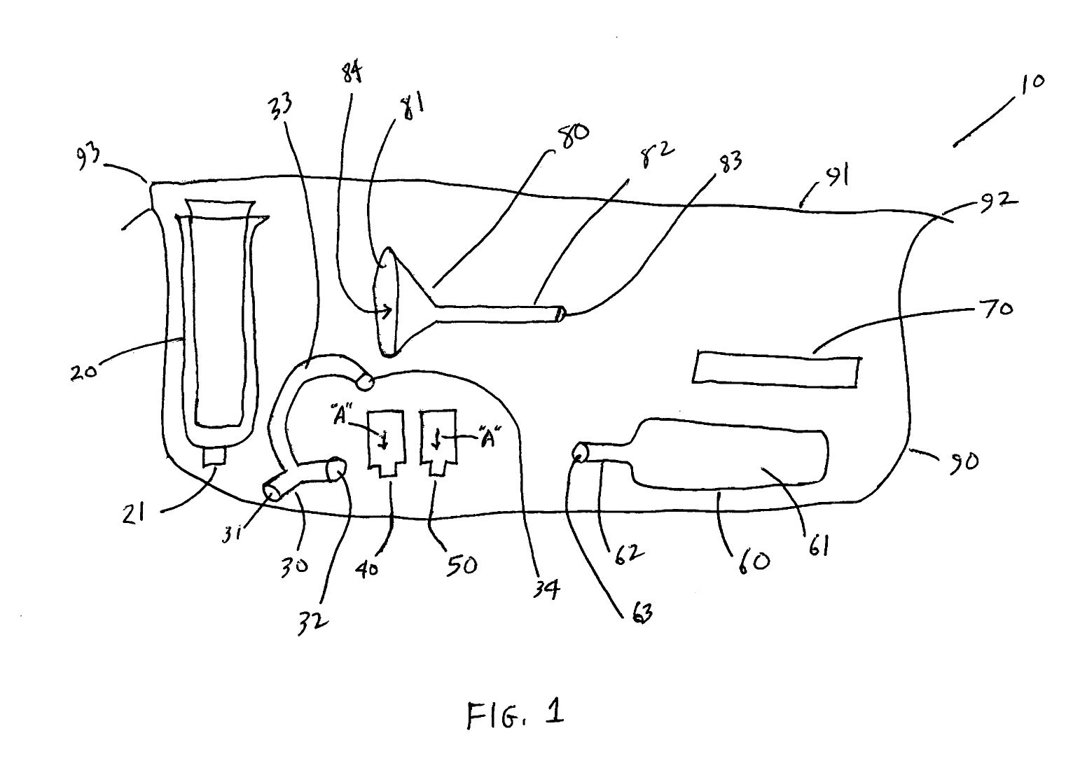

An exemplary kit comprising a packaging material, a syringe, a three-way connector, two one-way valves, a balloon, and an attachment member for attaching a neck portion of the balloon to a tubular shaft of a trocar is shown in FIG. 1.

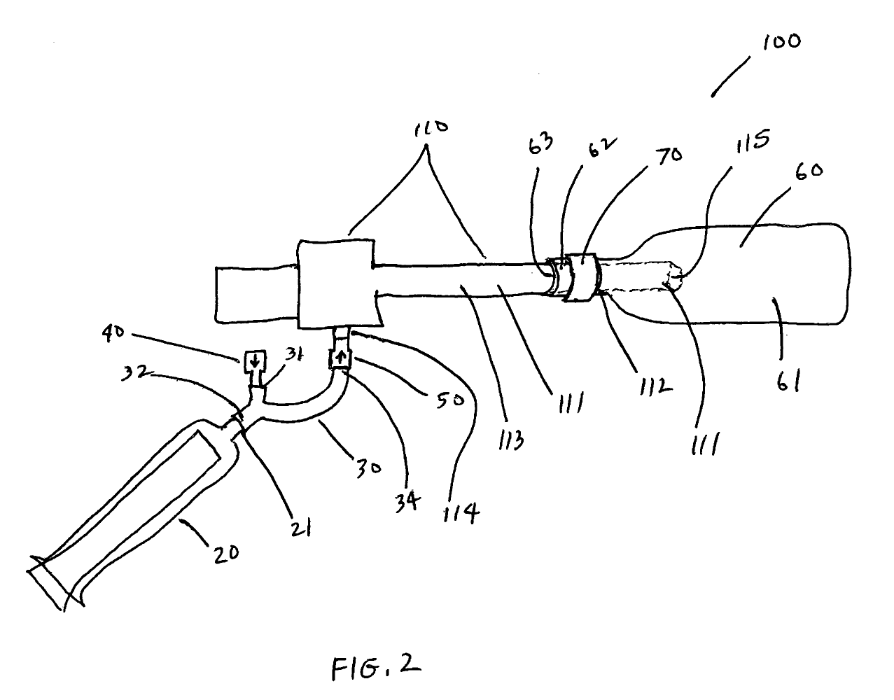

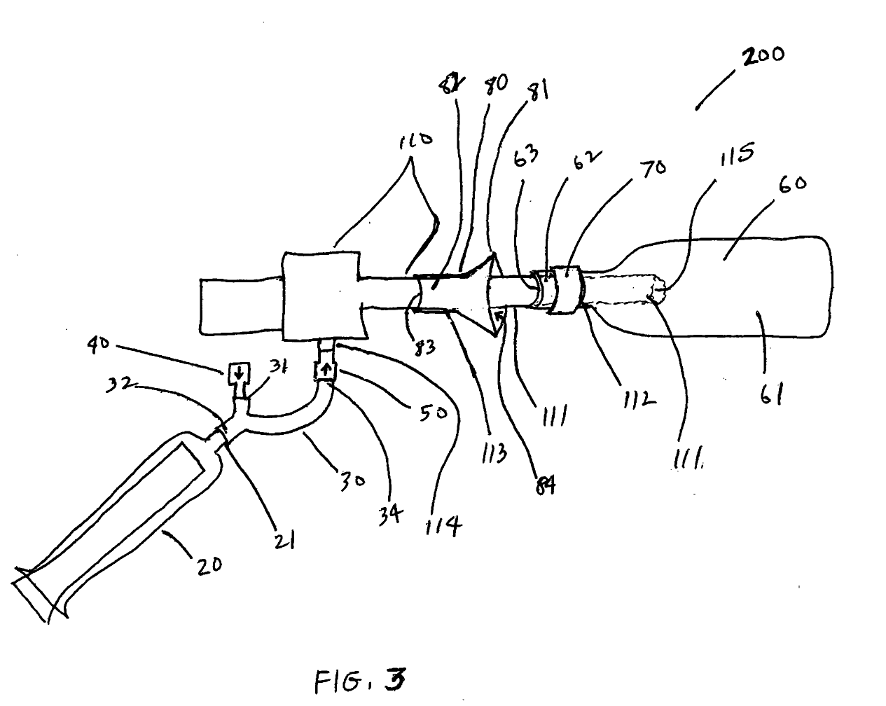

As shown in FIG. 1, exemplary kit 10 comprises syringe 20 having syringe outlet 21; three-way connector 30 having first inlet 31, second inlet 32, third inlet 34, and flexible tubular portion 33; one-way valves 40 and 50 having a fluid flow in a one-way direction as indicated by arrows “A”; balloon 60 having an inflatable body portion 61, a cylindrical neck portion 62 integrally attached to and extending from inflatable body portion 61, and an open end 63; an attachment member 70 capable of attaching neck portion 62 of balloon 60 to a tubular shaft of a trocar (see, for example, tubular shaft 111 of trocar 110 in FIGS. 2-3); an optional trocar seal 80 having a funnel shape with an open conical-shaped end portion 81 and a cylindrical sleeve portion 82 integrally attached to and extending from conical-shaped end portion 81); and packaging material 90.

As described above, kits of the present invention may comprise a number of components. A description of some of the suitable exemplary components is provided below.

### I. Kit Components

The kits of the present invention may comprise, but are not limited to, one or more of the following exemplary components.

A. Connectors

The kits of the present invention comprise at least one connector capable of connecting a fluid supply to a commercially available trocar. The fluid supply may be an air supply or a water supply or some other fluid supply (e.g., a saline solution supply). For example, the connector may comprise a conduit or hose for connecting an air supply or a water supply to a commercially available trocar.

In one exemplary embodiment, the connector comprises a two-way connector capable of connecting a fluid supply to a commercially available trocar. In a further exemplary embodiment, the connector comprises a three-way connector capable of connecting a fluid supply to a commercially available trocar, and providing an additional opening or port for an assembly containing the three-way connector. Such a three-way connector is shown as exemplary three-way connector 30 in FIG. 1.

Any commercially available connector may be used in the kits of the present invention. The dimensions of the connectors (i.e., diameter, length of tubing component, etc.) are not limited, although typically, the connector has a connector inside diameter of less about 10 millimeters (mm.). In one exemplary embodiment, the connector comprises a three-way connector having a connector inside diameter of about 5 mm.

Connectors, including three-way connectors, suitable for use in the present invention include, but are not limited to, connectors commercially available from Warner Instruments (Hamden, Conn.) or other suppliers, and commonly referred to as a “stopcock”. Desirably, the connector has LUER LOCK™ connections that match the connection of a fluid-supplying device, such as a syringe, and any other component attached to the connector. One particularly suitable connector comprises a three-way connector (or 3-way stopcock) with an extension line or flexible tubular portion such as flexible tubular portion 33 shown in FIG. 1.

Although the kits of the present invention typically contain a single connector, it should be noted that kits of the present invention may contain one or more connectors in a single kit.

B. Balloons

The kits of the present invention also comprise a balloon such as exemplary balloon 60 shown in FIG. 1. Any commercially available inflatable balloon may be used in the kits of the present invention. The size of the balloon is not limited, although typically the balloon has an inflatable volume capacity of up to about 3000 milliliters (ml.). In one exemplary embodiment, the balloon comprises a balloon having an inflatable volume capacity of about 2000 ml.

Balloons suitable for use in the present invention include, but are not limited to, balloons manufactured by Microtek Medical, Inc. (Alpharetta, Ga.). Such balloons are typically formed from a latex rubber material or a similar material, such as synthetic polyisoprene. The resulting balloon, when inflated, provides a semi-transparent view through the balloon material without undesirable glare during a surgical procedure.

In one exemplary embodiment, the latex balloon has the following dimensions: a neck portion length of about 20 mm., a neck portion inner diameter of about 11 mm., a body portion length of about 100 mm., a maximum body portion inner diameter (in a non-inflated state) of about 30 mm., and a wall thickness of about 0.25 mm.

Although the kits of the present invention typically contain a single balloon, it should be noted that kits of the present invention may contain one or more balloons in a single kit.

C. Attachment Members

The kits of the present invention may also comprise one or more attachment members such as exemplary attachment member 70 shown in FIG. 1. Typically, the attachment member comprises an adhesive tape, although other types of attachment members may be used in the present invention as long as the attachment member is capable of attaching the neck portion of a balloon (or the cylindrical sleeve portion of a trocar seal) to a tubular shaft of a trocar device.

The attachment member can (i) be fixed onto the neck of the balloon (or a cylindrical sleeve portion of a trocar seal (described below)) or (ii) be present as a separate component that is fixed onto the neck of the balloon (or the cylindrical sleeve portion of a trocar seal) after applying the balloon (and/or trocar seal) over a portion of a tubular shaft of a trocar.

Adhesive tapes suitable for use in the present invention include, but are not limited to, adhesive tapes commercially available from The 3M Company (St. Paul, Minn.). In one exemplary embodiment, the adhesive tape comprises 3M PVC Single Coated Foam Medical Tape No. 9781.

Although the kits of the present invention typically contain up to two attachment members, it should be noted that kits of the present invention may contain any number of attachment members (e.g., pieces of tape) in a single kit.

D. Fluid-Supplying Devices

The kits of the present invention may further comprise at least one fluid-supplying device. Suitable fluid-supplying devices include, but are not limited to, syringes, collapsible bulbs (e.g., bulbs used in combination with pipettes), and a portable pump.

In one desired embodiment, the fluid-supplying device comprises a syringe such as exemplary syringe 20 shown in FIG. 1. Any commercially available syringe may be used in the kits of the present invention. The size of the syringe is not limited, although typically, the syringe has a volume of less about 100 milliliters (ml.). In one exemplary embodiment, the syringe comprises a syringe having a volume of about 60 ml.

Syringes suitable for use in the present invention include, but are not limited to, syringes commercially available from Neogen Corporation (Lansing, Mich.) under the trade designation LUER LOCK™. One particularly suitable syringe comprises a 60 ml. LUER LOCK™ syringe, Item No. 9268.

Although the kits of the present invention typically contain no one fluid-supplying device or a single fluid-supplying device such as a syringe, it should be noted that kits of the present invention may contain one or more one fluid-supplying devices in a single kit.

E. One-Way Valves

The kits of the present invention may further comprise one or more one-way valves such as exemplary one-way valves 40 and 50 shown in FIG. 1. Any commercially available one-way valve may be used in the kits of the present invention. The size of the one-way valves is not limited, although typically, the one-way valves have a valve inside diameter of less about 10 millimeters (mm.). In one exemplary embodiment, the one-way valves comprise one-way valves having a valve inside diameter of about 5 mm.

One-way valves suitable for use in the present invention include, but are not limited to, one-way valves commercially available from Codan US (Santa Ana, Calif.) under the trade designation R-LOCK™. One particularly suitable one-way valve is R-LOCK™ one-way valve, Item No. 16.5250.

Although the kits of the present invention typically contain up to two one-way valves, it should be noted that kits of the present invention may contain any number of one-way valves in a single kit.

F. Trocar Seals

The kits of the present invention may further comprise an optional trocar seal such as exemplary trocar seal 80 shown in FIG. 1. Any commercially available trocar seal may be used in the kits of the present invention. The size of the trocar seal is not limited, although typically the trocar seal has a cylindrical sleeve portion with an inner diameter of less than about 20 mm. and a maximum diameter (i.e., the conical-shaped end portion) of less than about 80 mm.

Trocar seals suitable for use in the present invention include, but are not limited to, trocar seals manufactured by Microtek Medical, Inc. (Alpharetta, Ga.) from a latex rubber material or a similar material, such as synthetic polyisoprene. The resulting trocar seal provides an air-tight seal between the tubular shaft of a trocar and a patient's body. When in use, at least a portion of the trocar seal (e.g., at least a portion of the conical-shaped end portion of the trocar seal) is positioned inside the patient's body to minimize air leakage from within the patient's body.

In one exemplary embodiment, the trocar seal has a graduated cylindrical sleeve portion, namely, an end cylindrical sleeve portion and an intermediate cylindrical sleeve portion positioned between the end cylindrical sleeve portion and the conical-shaped end portion. In this embodiment, the latex trocar seal may have the following dimensions: an end cylindrical sleeve portion length of about 15 mm.; an end cylindrical sleeve portion inner diameter of about 11 mm. (or about 9 mm., or about 5 mm.); an intermediate cylindrical sleeve portion length of about 30 mm.; an intermediate cylindrical sleeve portion inner diameter of about 14 mm. (or about 12 mm., or about 8 mm.); a conical-shaped end portion length of about 30 mm.; a maximum conical-shaped end portion inner diameter of about 60 mm., and a wall thickness of about 0.25 mm.

Although the kits of the present invention typically contain no trocar seal or a single trocar seal, it should be noted that kits of the present invention may contain one or more trocar seals in a single kit.

G. Packaging Material

The kit may further comprise a packaging material to house the above-described components, such as packaging material 11. Typically, packaging material 11 comprises a disposable, transparent polymeric film material such as polyethylene film. However, any other packaging material may be used in the present invention including, but not limited to, paper packaging materials.

The packaging material may be a sealable or resealable packaging material. For example, exemplary packaging material 90 shown in FIG. 1 may have a sealable or resealable component extending along a line from point 92 to point 93. Suitable sealable or resealable components include, but are not limited to, an adhesive seal, a zip-lock type seal, or a hook and loop material.

In one exemplary embodiment of the present invention, the packaging material comprises a single-use, peel-open pouch. The peel-open pouch has a heat-sealed opening that can be opened by separating adjacent pouch components adjacent the opening.

### II. Methods of Making Kits

The above-described kits may be produced by obtaining and/or manufacturing each of the above-described components and packaging the components in a packaging material as described above. In one exemplary embodiment of the present invention, the method of making a kit comprises the step of placing one or more of the above-described kit components into a transparent packaging material, and sealing the transparent packaging material.

In one exemplary embodiment of the present invention, the kit is prepared using conventional techniques in order to produce a sterile kit, suitable for use in an operating room setting. Suitable sterilization methods include, but are not limited to, ethylene oxide (ETO) gas sterilization techniques.

### III. Methods of Using the Kits

The present invention is also directed to methods of using the above-described kits. In one exemplary method of using the above-described kits, the method comprises a method of preparing for a surgical procedure, wherein the method comprises the steps of providing a kit comprising a packaging material and components sized to fit within the packaging material, wherein the components comprise a connector that can be used to connect a fluid supply to a trocar, and a balloon; providing a trocar having a tubular shaft, wherein the tubular shaft has an outer surface suitable for excepting a neck portion of the balloon; removing the components from the packaging material; and assembling the components with the trocar so as to form a device suitable for pumping a fluid into the balloon so as to inflate the balloon.

In further exemplary embodiments, the method of using the above-described kits comprises the above-described steps, wherein the components comprise one or more additional components such as a first attachment member capable of attaching a neck portion of the balloon to a tubular shaft of a trocar, a fluid-supplying device, one or more one-way valves, a trocar seal, and an additional attachment member capable of attaching a cylindrical sleeve portion of a trocar seal to a tubular shaft of a trocar.

Any of the above-described methods of using the above-described kits results in a trocar assembly. One exemplary trocar assembly resulting from one of the above-described methods of using a kit is shown in FIG. 2.

As shown in FIG. 2, exemplary trocar assembly 100 comprising trocar 110 having a tubular shaft 111 and a trocar inlet 114. Neck portion 62 of balloon 60 extends over a first portion 112 of tubular shaft 111 of trocar 110. Balloon 60 is positioned over tubular shaft 111 as shown in FIG. 2 by inserting an end of tubular shaft 111 into opening 63 of balloon 60. Once balloon 60 is positioned over tubular shaft 111, attachment member 70 (e.g., an adhesive tape) is positioned over neck portion 62 of balloon 60 to secure neck portion 62 of balloon 62 to first portion 112 of tubular shaft 111. Syringe 20 is connected to first inlet 32 of three-way connector 30. First one-way valve 40 is connected to second inlet 31 of three-way connector 30 so that fluid (e.g., air) can not flow from second inlet 31 through first one-way valve 40. Second one-way valve 50 is positioned between third inlet 34 of three-way connector 30 and trocar inlet 114. Trocar inlet 114 is in fluid communication with interior channel 115 of tubular shaft 111 so that fluid (e.g., air) can not flow from trocar inlet 114 through second one-way valve 50.

Once exemplary trocar assembly 100 is fully assembled as shown in FIG. 2, syringe 20 may be pumped to force a fluid (such as air, water or a saline solution) through second one-way valve 50 and into balloon 60 to inflate balloon 60 a desired amount. First one-way valve 40 may also be used to input a second fluid into the trocar assembly.

In another exemplary embodiment, the above-described method of preparing for a surgical procedure further comprises the steps of providing a trocar seal having a funnel shape with an open conical-shaped end portion and a cylindrical sleeve portion integrally attached to and extending from the concial-shaped end portion; and positioning the cylindrical sleeve portion of the trocar seal over a portion of the tubular shaft of the trocar so that the conical-shaped end portion of the trocar seal extends toward the balloon. This exemplary method results in a trocar assembly similar to the exemplary trocar assembly shown in FIG. 3.

As shown in FIG. 3, exemplary trocar assembly 200 comprises each of the components described with reference to trocar assembly 100 of FIG. 2. In addition, trocar assembly 200 comprises trocar seal 80 such that cylindrical sleeve portion 82 of trocar seal 80 extends over second portion 113 of tubular shaft 111 of trocar 110. Trocar seal 80 is positioned over tubular shaft 111 as shown in FIG. 2 by inserting an end of tubular shaft 111 into opening 83 of trocar seal 80. Once trocar seal 80 is positioned over second portion 113 of tubular shaft 111, balloon 60 is positioned over first portion 112 of tubular shaft 111 as described above in trocar assembly 100. As shown in FIG. 3, conical-shaped end portion 81 and open end 84 of trocar seal 80 extend toward balloon 60.

As with trocar assembly 100 described above, once exemplary trocar assembly 200 is fully assembled as shown in FIG. 3, syringe 20 may be pumped to force a fluid (such as air, water or a saline solution) through second one-way valve 50 and into balloon 60 to inflate balloon 60 a desired amount.

Trocar assemblies, such as exemplary trocar assemblies 100 and 200 shown in FIGS. 2-3, are particular useful in surgical procedures such as laparoscopic surgical procedures. Trocar assemblies similar to exemplary trocar assembly 200 provide additional protection against possible air leakage between the tubular shaft of the trocar and a patient's body during a surgical procedure, such as a laparoscopic surgical procedure. In one exemplary procedure, at least a portion of the conical-shaped end portion of the trocar seal (i.e., conical-shaped end portion 81 of trocar seal 80) is positioned within a patient's body, and serves to minimize air (or other fluid) leakage during a surgical procedure.

### IV. Methods of Doing Business

The present invention is also directed to methods of doing business related to the above-described kits. In one exemplary embodiment, the method of doing business comprises the steps of providing a kit comprising a packaging material and components sized to fit within the packaging material, wherein the components comprise a connector that can be used to connect a fluid supply to a trocar, and a balloon; wherein the method provides an alternative to commercially available trocar assemblies comprising a preassembled trocar and balloon combination.

In further exemplary embodiments, the method of doing business comprises the above-described steps, wherein the components comprises one or more additional components such as a first attachment member capable of attaching a neck portion of the balloon to a tubular shaft of a trocar, a fluid-supplying device, one or more one-way valves, a trocar seal, and an additional attachment member capable of attaching a cylindrical sleeve portion of a trocar seal to a tubular shaft of a trocar.

In any of the above-described exemplary methods, the kits can be used with any commercially available trocar. Further, the providing step of any of the above-described methods may comprise offering the kit for sale.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

### Preparation of a Kit

An exemplary kit as shown in FIG. 1 was prepared. Each of the kit components shown in exemplary kit 10 were placed into a transparent zip-lock bag, and temporarily sealed.

### EXAMPLE 2

### Use of a Kit in a Surgical Procedure

The exemplary kit of Example 1 was used to prepare a trocar assembly as shown in FIG. 3.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A kit for use during a surgical procedure, said kit comprising a packaging material and components sized to fit within the packaging material, said components comprising at least:
a connector that can be used to connect a fluid supply to a trocar; and
a balloon.

2. The kit of claim 1, wherein the components further comprise:
a first attachment member capable of attaching a neck portion of the balloon to a tubular shaft of a trocar.

3. The kit of claim 2, wherein the components further comprise:
a syringe; and
a first one-way valve.

4. The kit of claim 2, wherein the connector comprises a three-way connector, and the components further comprise:
a syringe; and
two one-way valves.

5. The kit of any one of claims 1, wherein the components further comprise a trocar seal having a funnel shape with an open conical-shaped end portion and a cylindrical sleeve portion integrally attached to and extending from the concial-shaped end portion.

6. The kit of claim 5, wherein the components further comprise a second attachment member capable of attaching the cylindrical sleeve portion of the trocar seal to a tubular shaft of a trocar.

7. The kit of claim 6, wherein the first and second attachment members comprise an adhesive tape.

8. The kit of claim 4, wherein the three-way connector comprises a flexible tubular material extending from at least one inlet of the three-way connector.

9. The kit of claim 1, wherein the balloon comprises an inflatable body portion and a cylindrical neck portion integrally attached to and extending from the inflatable body portion.

10. The kit of claim 1, wherein the balloon comprises a rubber latex material.

11. The kit of claim 5, wherein each of the balloon and the trocar seal comprises a rubber latex material.

12. The kit of claim 1, wherein the components are positioned with the packaging material, the packaging material being in a sealed configuration.

13. The kit of claim 12, wherein the packaging material comprises a transparent film material so that the components can be viewed through the packaging material.

14. The kit of claim 1 in combination with a trocar.

15. The kit of claim 14, wherein the trocar comprises any commercially available trocar that does not have a balloon preassembled therewith.

16. A trocar assembly comprising the components of the kit of claim 4 in combination with a trocar, wherein the neck portion of the balloon extends over a portion of a tubular shaft of the trocar; the attachment member comprises an adhesive tape and is positioned over the neck portion of the balloon so as to surround the neck portion of the balloon and the portion of the tubular shaft covered by the balloon; the syringe is connected to a first inlet of the three-way connector; a first one-way valve is connected to a second inlet of the three-way connector so that fluid can not flow from the second inlet through the first one-way valve; and a second one-way valve is positioned between a third inlet of the three-way connector and a trocar inlet, said trocar inlet being in fluid communication with an interior channel of the tubular shaft so that fluid can not flow from the trocar inlet through the second one-way valve.

17. A trocar assembly comprising the components of the kit of claim 5 in combination with a trocar, wherein the neck portion of the balloon extends over a first portion of a tubular shaft of the trocar; the attachment member comprises an adhesive tape and is positioned over the neck portion of the balloon so as to surround the neck portion of the balloon and the first portion of the tubular shaft covered by the balloon; the syringe is connected to a first inlet of the three-way connector; a first one-way valve is connected to a second inlet of the three-way connector so that fluid can not flow from the second inlet through the first one-way valve; a second one-way valve is positioned between a third inlet of the three-way connector and a trocar inlet, said trocar inlet being in fluid communication with an interior channel of the tubular shaft so that fluid can not flow from the trocar inlet through the second one-way valve; and the cylindrical sleeve portion of the trocar seal extends over a second portion of the tubular shaft of the trocar so that the conical-shaped end portion of the trocar seal extends toward the balloon.

18. A method of preparing for a surgical procedure, said method comprising the steps of:
providing the kit of claim 1;
removing the components from the packaging material; and
assembling the components with a trocar so as to form a device suitable for pumping a fluid into the balloon so as to inflate the balloon.

19. A method of preparing for a surgical procedure, said method comprising the steps of:
providing a kit comprising a packaging material and components sized to fit within the packaging material, said components comprising at least:
a connector that can be used to connect a fluid supply to a trocar; and
a balloon;
providing a trocar having a tubular shaft, wherein the tubular shaft has an outer surface suitable for excepting the neck portion of the balloon;
removing the components from the packaging material; and
assembling the components with the trocar so as to form a device suitable for pumping a fluid into the balloon so as to inflate the balloon.

20. The method of claim 19, wherein the components further comprise:
a first attachment member capable of attaching a neck portion of the balloon to a tubular shaft of a trocar;
a syringe;
two one-way valves;
a trocar seal having a funnel shape with an open conical-shaped end portion and a cylindrical sleeve portion integrally attached to and extending from the concial-shaped end portion; and
a second attachment member capable of attaching the cylindrical sleeve portion of the trocar seal to a tubular shaft of a trocar.

21. The method of any one of claims 19, further comprising:
providing a trocar seal having a funnel shape with an open conical-shaped end portion and a cylindrical sleeve portion integrally attached to and extending from the concial-shaped end portion; and
positioning the cylindrical sleeve portion of the trocar seal over a portion of the tubular shaft of the trocar so that the conical-shaped end portion of the trocar seal extends toward the balloon.

22. A method of doing business, said method comprising the steps of:
providing a kit comprising a packaging material and components sized to fit within the packaging material, said components comprising at least:
a connector that can be used to connect a fluid supply to a trocar; and
a balloon;
wherein said method provides an alternative to commercially available trocar assemblies comprising a pre-assembled trocar/balloon combination.

23. The method of claim 22, wherein the components further comprise:
a first attachment member capable of attaching a neck portion of the balloon to a tubular shaft of a trocar;
a syringe;
two one-way valves;
a trocar seal having a funnel shape with an open conical-shaped end portion and a cylindrical sleeve portion integrally attached to and extending from the concial-shaped end portion; and
a second attachment member capable of attaching the cylindrical sleeve portion of the trocar seal to a tubular shaft of a trocar.

24. The method of claim 23, wherein each of the first and second attachment members comprises an adhesive tape.

25. The method of any one of claims 22, wherein the kit is used in combination with any commercially available trocar.

26. The method of any one of claims 22, wherein the providing step comprises offering the kit for sale.
